(19) United States
(12) Patent Application Publication
(10) Pub. No.: US 2009/0099596 A1
(43) Pub. Date: Apr. 16, 2009

(54) Closure device for left atrial appendage

(75) Inventors: James F. McGuckin, JR., Radnor, PA (US); David M. Schaller, Wallingford, PA (US); James Erich Bressler, Langhorne, PA (US)

Correspondence Address: Neil D. Gershon, 1011 High Ridge Road, Stamford, CT, 06905, US

(73) Assignee: Rex Medical

(21) Appl. No.: 12/151,790

(22) Filed: May 9, 2008

Related U.S. Application Data
(60) Provisional application No. 60/932,448, filed on May 31, 2007.

(51) Int. Cl. A61B 17/06; A61B 17/08

(52) U.S. Cl. 606/216; 606/213

(57) ABSTRACT

A device for closing the left atrial appendage of a patient comprising a retention member composed of a shape memory material and a mesh material supported by the retention member. The retention member has a first elongated configuration for delivery and a second expanded configuration for placement within the left atrial appendage. The mesh is configured to block blot clot migration from the appendage. In the second configuration the retention member moves toward a shape memory position. The retention member has a plurality of appendage wall engagement members to secure the retention member to the appendage.

This application claims priority from provisional application. Ser. No. 60/932,448 filed May 31, 2007, the entire contents of which is incorporated herein by reference.

### BACKGROUND

1. Technical Field

This application relates to a closure device and more particularly to a device for closing the left atrial appendage of the heart.

2. Background of Related Art

The atrial appendage is a small muscular pouch or cavity attached to the atrium of the heart. The left atrial appendage (LAA) is connected to the wall of the left atrium between the mitral valve and the left pulmonary vein. In proper functioning, the left atrial appendage contracts with the rest of the left atrium during a heart cycle, ensuring regular flow of blood.

Atrial fibrillation is the irregular and randomized contraction of the atrium working independently of the ventricles. This resulting rapid and chaotic heartbeat produces irregular and turbulent blood flow in the vascular system, resulting in the left atrial appendage not contracting regularly with the left atrium. Consequently, the blood can become stagnant and pool in the appendage, resulting in blood clot formation in the appendage. If the blood clot enters the left ventricle it can enter the cerebral vascular system and cause embolic stroke, resulting in disability and even death.

One approach to treatment is the administration of medications to break up the blood clots. However, these blood thinning medications are expensive, increase the risk of bleeding and could have adverse side effects. Another approach is to perform invasive surgery to close off the appendage to contain the blood clot within the appendage. Such invasive open heart surgery is time consuming, traumatic to the patient, increases patient risk and recovery time, and increases costs as extended hospital stays are required.

It is therefore recognized that a minimally invasive approach to closing off the appendage to prevent the migration of blood clots into the ventricle and cranial circulation would be beneficial. These devices, however, need to meet several criteria.

Such minimally invasive devices need to be collapsible to a small enough dimension to enable delivery through a small incision while being expandable to a sufficiently large dimension with sufficient stability to ensure sealing of the appendage is maintained. These devices also need to be atraumatic. Further, the size of the appendage can vary among patients and therefore the devices need to be expandable to the appropriate size to close off the appendage.

There have been several attempts in the prior art to provide minimally invasive appendage closure devices. For example, in U.S. Pat. No. 6,488,689, a capture loop or clip is placed around the appendage to hold the appendage closed. These devices can be traumatic to the vascular structure. The Amplatzer occluder marketed by AGA Medical, provides for stent like expansion within a balloon. However, the diameter of expansion is not controllable and the collapsed configuration is relatively large, disadvantageously increasing the profile for insertion. In U.S. Pat. No. 6,152,144, an occluding member having an outer rim and a thin mesh barrier to provide a seal is placed at the opening of the appendage. Radially extending shape memory members extend from the shaft to anchor the device. An expandable anchoring member is also disclosed. In another embodiment, an occlusive coil having a random configuration is placed in the appendage to induce clot. U.S. Pat. Nos. 6,551,303 and 6,652,555 disclose a membrane placed across the ostium of the atrial appendage to prevent blood from entering. Various mechanisms such as shape memory prongs, anchors, springs and struts function to retain the membrane. These devices, however, suffer from various deficiencies.

Therefore, there is a need for an improved closure device for the left atrial appendage which will effectively block blood clot migration, remain securely retained within the appendage, and have a reduced delivery profile to minimize the surgical incision and facilitate passage through the vascular system.

### SUMMARY

The present invention overcomes the problems and deficiencies of the prior art. The present invention provides a device for closing the left atrial appendage of a patient comprising a retention member composed of a shape memory material and a mesh material supported by the retention member. The retention member has a first elongated configuration for delivery and a second expanded configuration for placement within the left atrial appendage. The mesh is configured to block blot clot migration from the appendage. In the second configuration the retention member moves toward a shape memory position. The retention member has at least one appendage wall engagement member to secure the retention member to the appendage.

In one embodiment, the mesh is attached to an outer surface of the retention member. In another embodiment, the retention member has a plurality of struts defining a space and the mesh fills a substantial region of the space. In another embodiment, the mesh is a strip of material connected to the retention member and spanning an opening of the retention member. The mesh in this embodiment is preferably positioned at a region adjacent the wall engagement members.

The present invention also provides a device for occluding the left atrial appendage comprising a tube laser cut to form a series of struts, the tube having a first elongated configuration for delivery and a second configuration for placement. In the second configuration, the tube has an expanded configuration. The struts extend outwardly so that a distal region of the struts has a greater dimension and the struts define a space therebetween. A mesh material is supported by the struts and provides a blocking member to block blot clot migration from the appendage. In one embodiment, the mesh material fills a substantial area of the space between the struts.

In another embodiment, the mesh material is in the form of a narrow strip attached to one or more of the struts. In another embodiment, the mesh is attached to an outer surface of the struts and extends across a proximal region of the device.

A method for left atrial appendage occlusion is also provided comprising the steps of inserting into the left atrial appendage a sheath containing a retention member having a plurality of struts in a reduced profile position, exposing the retention member from the sheath to enable it to expand to engage a wall of the left atrial appendage, subsequently inserting mesh material in situ within a space between the plurality of struts and withdrawing the sheath to leave the retention member in the left atrial appendage so the mesh material fills the space within the retention member to block blood clot migration.

Preferably, the retention member has a plurality of shape memory struts and the step of exposing the retention member enables the struts to move toward a shape memorized position.

### BRIEF DESCRIPTION OF THE DRAWINGS

Preferred embodiment(s) of the present disclosure are described herein with reference to the drawings wherein:

FIG. 1 is a perspective view of the retention member of the left atrial appendage closure (or occlusion) device of the present invention shown in the collapsed position for delivery;

FIG. 2 is a transverse cross-sectional view taken along line 2-2 of FIG. 1;

FIG. 3 is a cross-sectional view taking along lines 3-3 of FIG. 1 showing a portion of the retention member within a delivery sheath;

FIG. 4 is a perspective view showing the retention member with mesh contained therein in the expanded condition:

FIGS. 5 and 6 are side and front views of the device of FIG. 4:

FIG. 7 is an anatomical view showing insertion of the closure device through the femoral vein of a patient to access the left atrial appendage;

FIG. 8 illustrates placement of the closure device in the left atrial appendage;

FIG. 9 is a close up view of the area of detail of FIG. 8;

FIG. 9A is a close up view similar to FIG. 9 except showing an alternate embodiment of the device;

FIG. 10 is a perspective view of an alternate embodiment of the closure device of the present invention wherein the device is oriented in a direction opposite to that of FIG. 9:

FIGS. 11 and 12 are perspective and side views, respectively, of another alternate embodiment of the device having a strip of mesh contained therein;

FIG. 13 is a side view of another alternate embodiment of the device having a mesh filling substantially the entire space between the struts;

FIG. 14 is a side view of yet another alternate embodiment of the device having a mesh positioned on the outside of the retention member; and

FIG. 15 is a perspective view of another alternate embodiment of the closure device having mesh supported by a wound wire.

### DETAILED DESCRIPTION OF PREFERRED EMBODIMENTS

Referring now in detail to the drawings where like reference numerals identify similar or like components throughout the several views, the present invention provides a closure device for closing or occluding the left atrial appendage (“LAA”). The device can be inserted minimally invasively. The device includes a securement (retention) member and mesh material. The securement member provides for attachment to the appendage wall as well as a support or retention member for the various embodiments of the mesh described below.

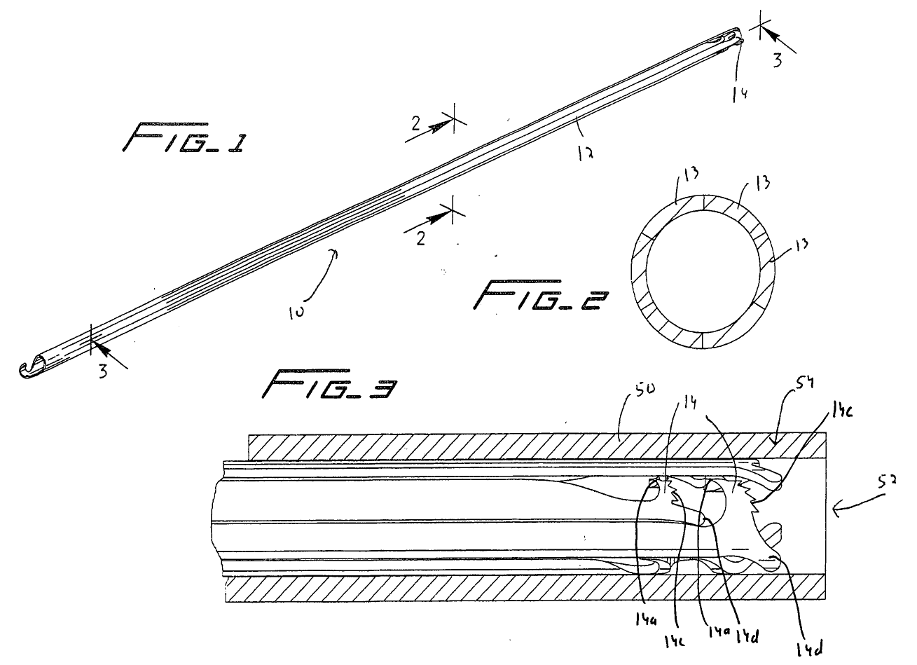

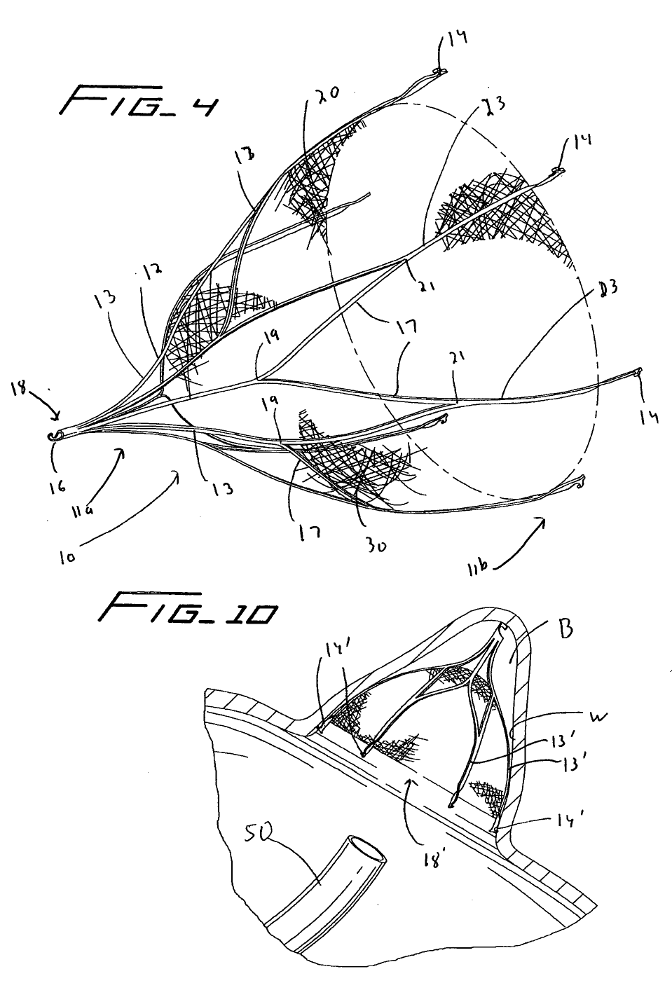

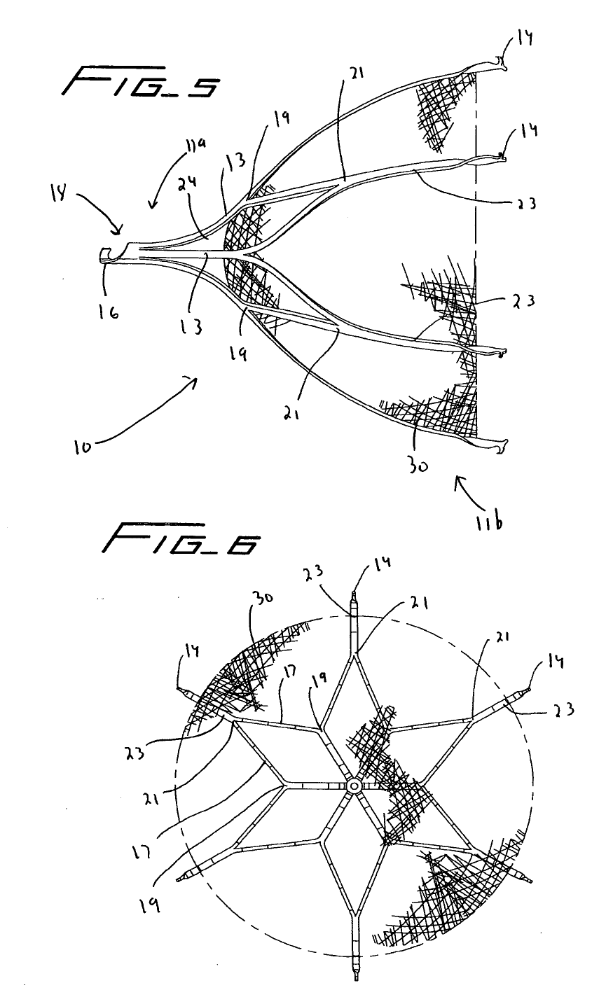

With initial reference to FIGS. 1-3 which show the closure device in the low profile delivery (collapsed) configuration for insertion and FIG. 4 which shows the closure device in the expanded configuration for deployment, the closure device 10 includes a securement or retention component (member) 12. The securement member 12 forms a containment member to receive the mesh therein and has engagement hooks 14 for engaging the wall to retain the securement member 12 within the appendage. Mesh 30 can be advanced into the member 12 in situ or alternatively can be positioned in the securement member 12 in the delivery position and then advanced together with the securement member 12 to block the LAA opening to prevent migration of blood clots from the appendage. The closure device is preferably formed from a laser cut tube, although other ways of forming the device are also contemplated. The mesh is not shown in FIGS. 1-3 for clarity.

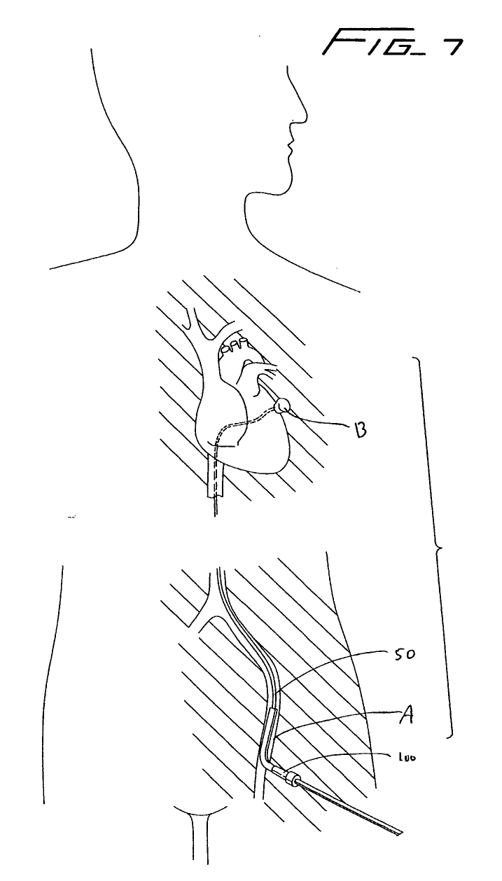

Turning to FIGS. 4-6 which illustrate the device 10 in the expanded (deployed) position, the retention component 12 is in the form of a bell shaped device with struts as described in detail with respect to the filter disclosed in patent application Ser. No. 10/889,429, filed Jul. 12, 2004 (the '429 application), the entire contents of which are incorporated herein by reference. The device has a proximal end 11a and a distal end 11b. The securement member 12 is preferably composed of shape memory material, such as Nitinol, with an austenitic shape memorized position illustrated in FIG. 4 and has a plurality of struts 13 emerging from apex 18 at proximal end 11a and terminating in wall engaging or retention hooks 14 at distal end 11b. In this embodiment, six struts are provided although a different number of struts is also contemplated. A retrieval hook 16 is positioned on the proximal end 11a to enable the device 10 to be grasped by a snare or other device and removed if desired. The struts 13 can be interconnected by interconnecting struts 17 which join adjacent struts. More specifically, the struts 13 preferably divide at region 19 into two connecting struts 17, angling away from each other, and then join at region 21 to form extending strut portions 23 terminating in hooks 14. The interconnecting struts 17 stiffen the device to enhance retention and increase the radial force. They also provide a more symmetric and uniform deployment. The hooks are configured to engage the appendage wall for maintaining the position of the device 10. The struts are preferably flared and create a distal opening and a space between the struts. For clarity, not all the identical parts are labeled throughout the drawings. It should be appreciated that materials other than Nitinol or shape memory are also contemplated.

The hooks 14 preferably extend substantially perpendicular from the strut and are preferably formed by torquing the struts so the hooks bend out of the plane. Preferably, a first set of hooks is larger than a second set of hooks. Preferably, when formed in a laser cut tube, the larger hooks are formed so that they occupy a region equivalent to the transverse dimension of two adjacent struts. Preferably, three smaller hooks and three larger hooks are provided in alternating arrangement in the embodiment utilizing six struts. The smaller hooks are preferably spaced axially with respect to each other and axially inwardly with respect to the larger hooks as in the filter hooks of the '429 application to minimize the collapsed profile (transverse dimension) of the filter when collapsed for insertion. The penetrating tips 14a (FIG. 3) penetrate the tissue to retain the device 10, and preferably point toward the proximal end 11a of the device.

Each of the hooks 14 has a series of teeth 14c respectively to engage the appendage wall to provide additional retention to prevent movement of the device. A heel 14d is provided which extends past the hook 14 to function as a stop to prevent the closure device from going through the wall. The angle of the heel 14d in the smaller hooks is less than the angle in the larger hooks to provide room for nesting of the hooks as shown in FIG. 3. For clarity, not all of the hooks are fully labeled.

The securement (retention) member 12 is maintained in a substantially straightened softer martensitic configuration within the delivery catheter or sheath 50 for delivery as shown in FIG. 3. The smaller hooks preferably nest within the larger hooks. Cold saline can be injected during delivery to maintain the struts 13 in this martensitic condition to facilitate exit from the distal opening 52 at the distal end portion 54 of catheter 50. When the struts 13 exit the delivery sheath (tube) 50, they are warmed by body temperature and move toward their illustrated memorized position as shown in FIGS. 4-6.

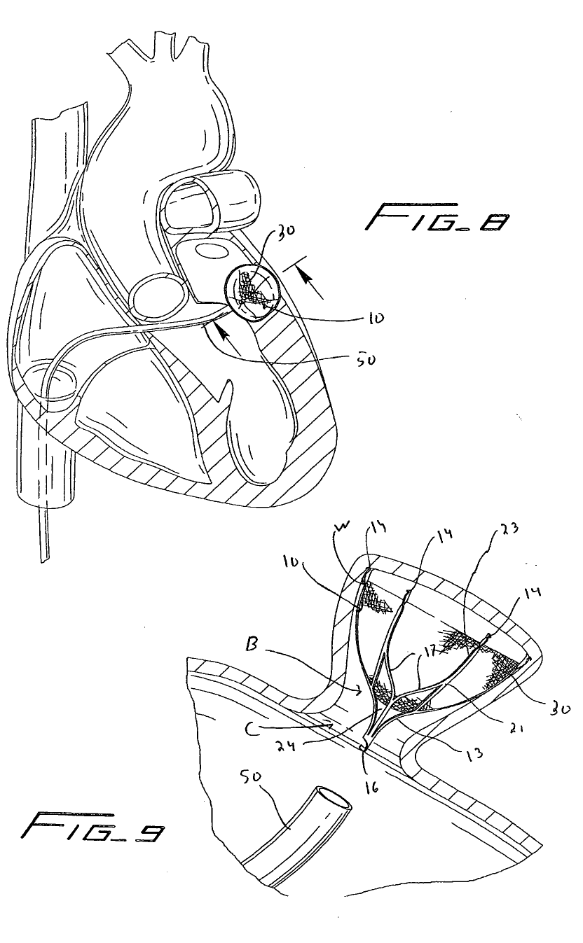

As shown in FIGS. 7-9, the device 10 is preferably inserted within delivery catheter 50 through the femoral vein A and advanced through the septum to access the left atrial appendage B. It is positioned in this embodiment with the distal end 11b further from the appendage opening (the retrieval hook 16 adjacent the appendage opening). When positioned in the appendage, the hooks 14 engage the wall to retain the device in the appendage.

The device 10 in the embodiment of FIGS. 1-7 has mesh material positioned within the retention member 12, filling substantially the entire region of the retention member 12. A small gap 24 can be left at the proximal region (see e.g. FIG. 5). However, in an alternate embodiment, the gap is filled in with mesh so the mesh fills more of the area between the struts as shown for example in FIG. 13 wherein mesh 62 fills substantially the entire space between the struts 63 of retention member 60 which is otherwise identical to retention member 12 of FIG. 1. The mesh is preferably in the form a tightly woven material to provide sufficiently small spaces to effectively block blood clot migration from the appendage.

The mesh can be delivered within the retention member 12 such that in the collapsed position the mesh is contained and compressed therein. After delivery, it would expand within the space of the retention member 12, i.e. within the space between the struts.

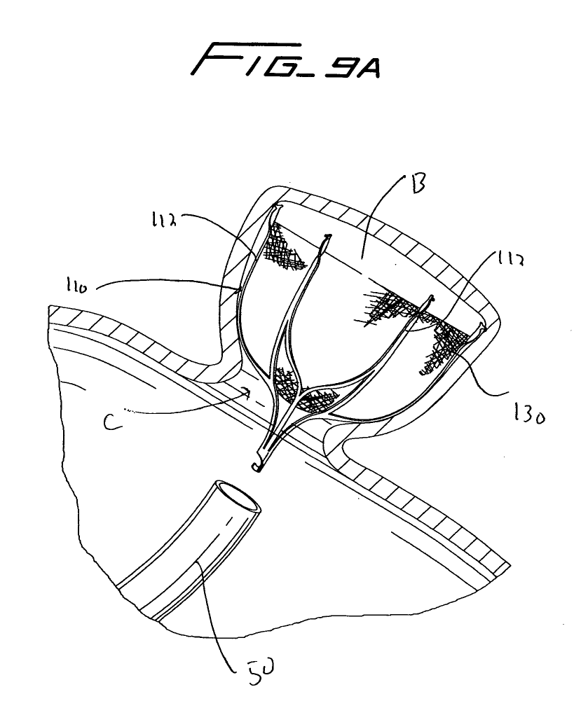

In an alternate embodiment, the retention member 12 would be placed within the appendage first, and then once in place, the mesh would be delivered through the opening of the device and within the space between the struts 13. This in situ delivery could occur in embodiments wherein the device 10 is implanted in an orientation opposite to that of FIG. 9, i.e. the opening 18′ between the struts 13′ would face in the other direction such that the hooks 14′ would be closer to the appendage opening as shown in FIG. 10.

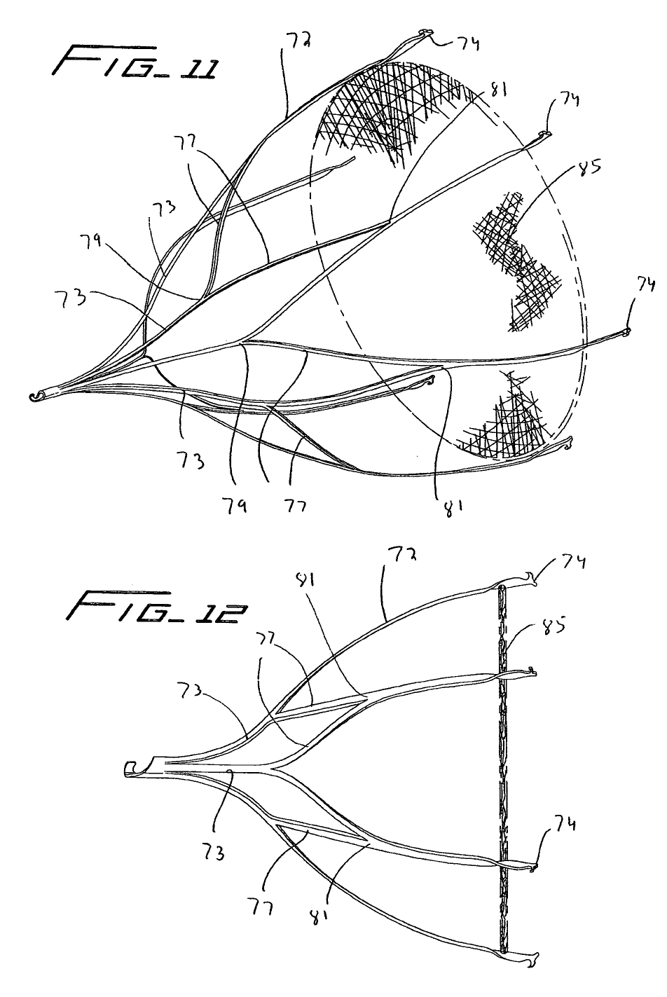

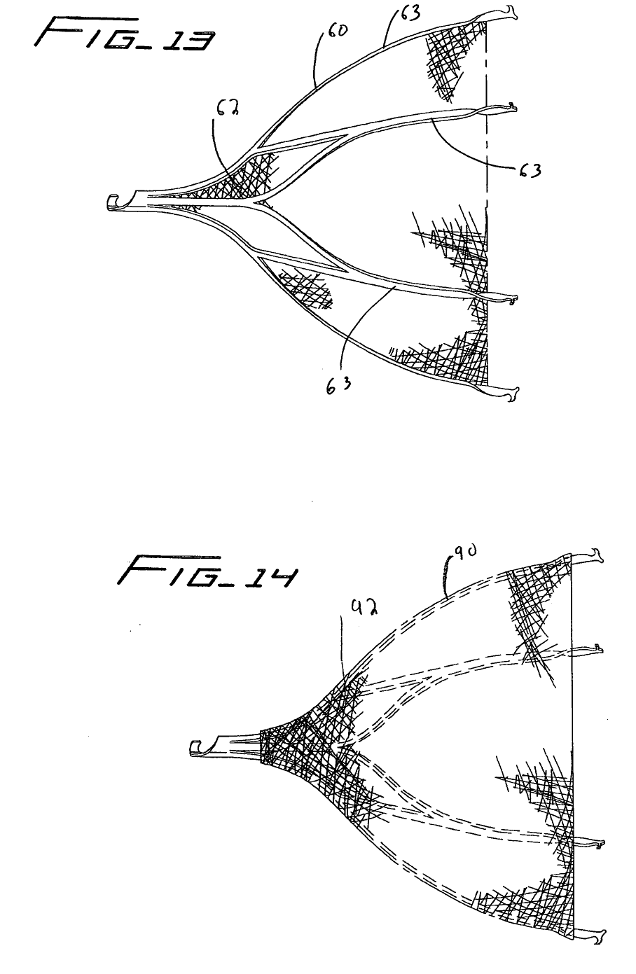

In an alternate embodiment, instead of the mesh filling the space between the struts, the mesh can be in the form of a narrow strip as shown in FIGS. 11 and 12. The mesh in this embodiment functions as a screen type blocking member. The securement (retention) member 72 is otherwise identical to securement member 12 of FIG. 1, e.g. struts 73 divide at region 79 into interconnecting struts 77, join at region 81 and terminate in vessel engaging hooks 74. The strip 85 of mesh would preferably be positioned slightly proximal of the hooks 74, e.g. at the region where the strut twists out of the plane so as not to interfere with the hooks. However, the mesh could be placed at other regions as long as it functions to effectively occlude the appendage, i.e. functions as a cover to prevent blood clot migration from the appendage. Although a thin strip of mesh is shown, other size blocking strips could also be provided. The mesh is shown attached to an inner surface of the struts but could alternatively be attached to the outer surface. It could be attached to one or more of the struts.

As noted above, although the securement member is shown inserted with the engaging hooks 14 within the appendage and the retrieval hook at the juncture with the atrium, it is also contemplated that the securement member be oriented in the opposite direction. This is shown for example in FIG. 10, wherein the vessel engaging hooks 14′ are at the opening of the appendage. In this version, the mesh can be inserted with the securement member or alternatively if desired can be delivered in situ within the opening 18′ between the struts 13′ in an already placed securement member.

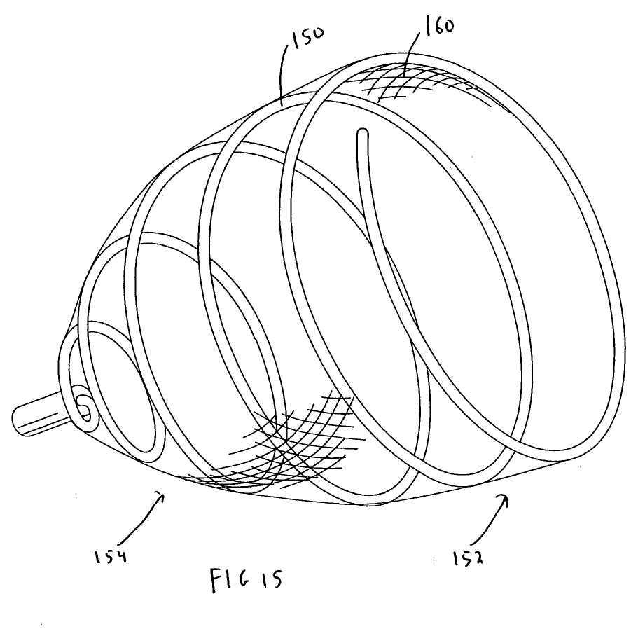

In the alternate embodiment of FIG. 14, the mesh 92 is positioned on the outside of securement member 90. In all other respects, securement member 90 is similar to securement member 12 of FIG. 4. In this embodiment, the mesh 92 is placed on an outer region, covering the outer surfaces of the strut and apex region and interposed between the struts and appendage wall when placed. Thus, the mesh functions as a sleeve which prevents passage of the clots as they would be captured within the sleeve or net-like device.

The mesh in the foregoing embodiments can be attached by various methods such as bonding, clamping, or suturing.

The method of placement of the closure device of the present invention will now be described for closing a left atrial appendage in conjunction with the embodiment of FIG. 1 by way of example with the mesh delivered in conjunction with the securement member (the other embodiments are inserted in a similar fashion). A delivery catheter 50 is inserted through an introducer sheath 100 in the femoral vein A and advanced through the septum to access the left atrial appendage B as shown in FIG. 7. For insertion, the securement member is in the collapsed position.

A pusher (not shown) is advanced distally at a proximal end of the catheter 50 to advance the device 10 from the catheter 50. As the struts are exposed, they are warmed by body temperature and return toward their shape memorized deployed position as shown in FIG. 9 to engage the appendage wall W. The extent they return to their fully memorized position will depend on the size of the appendage.

Preferably, the securement member will be positioned at the opening to the appendage and be substantially flush with the opening as shown in FIG. 9. Alternatively, a portion may extend past the opening into the atrium. For example, in the alternate embodiment of FIG. 9A, device 110 has struts 112 forming a wider base to conform to the shape of the appendage at the opening, with the mesh 130 extending up to the appendage opening C.

As can be appreciated, the device in the embodiments disclosed herein blocks the opening C in the appendage B to prevent migration of thrombus from the appendage into the atrium and left ventricle.

Note, the material inside or outside the securement member could be non-porous or porous. It could alternatively be made of pericardium, SIS, PET, PTFE, etc.

In the alternate embodiment of FIG. 15, a wound wire 150 provides a retention member for mesh 160. The wire as shown has a substantially conical configuration so the diameter (transverse dimension) at region 152 exceeds the diameter (transverse dimension) of region 154 and receives the mesh 160 inside. Alternatively, the mesh could be attached on the outside or a strip of mesh spanning the opening could be provided. The wire could have hooks, barbs or other surfaces to enhance retention in addition to the outward radial force against the appendage.

As can be appreciated, although described for closing the left atrial appendage of the heart, the closure device can also be used to embolize or occlude other conduits such as blood vessels, ureters of fistulas.

While the above description contains many specifics, those specifics should not be construed as limitations on the scope of the disclosure, but merely as exemplifications of preferred embodiments thereof. For example, other materials can be contained or mounted to the retention member to function to block blood clot migration from the left atrial appendage. Those skilled in the art will envision many other possible variations that are within the scope and spirit of the disclosure as defined by the claims appended hereto.

What is claimed is:

1. A device for closing the left atrial appendage of a patient comprising a retention member composed of a shape memory material and a mesh material supported by the retention member, the retention member having a first elongated configuration for delivery and a second expanded configuration for placement within the left atrial appendage, the mesh configured to block blood clot migration from the appendage, in the second configuration the retention member moving toward a shape memory position, the retention member having at least one appendage wall engagement members to secure the retention member to the appendage.

2. The device of claim 1, wherein the mesh is attached to an outer surface of the retention member.

3. The device of claim 1, wherein the mesh is positioned within the retention member.

4. The device of claim 1, wherein the retention member has a plurality of struts defining a space and the mesh fills a substantial region of the space.

5. The device of claim 1, wherein the mesh is a strip of material connected to the retention member and spanning an opening of the retention member.

6. The device of claim 5, wherein the mesh is positioned at a region adjacent the wall engagement members.

7. The device of claim 1, wherein the retention member has a plurality of struts forming a space therebetween and the mesh is positioned to span the space.

8. The device of claim 1, wherein the engagement members include a plurality of teeth.

9. The device of claim 1, wherein the retention member has a plurality of struts and the struts terminate in the engagement members.

10. The device of claim 1, wherein the retention member comprises a wound wire.

11. A device for occluding the left atrial appendage comprising a tube laser cut to form a series of struts, the tube having a first elongated configuration for delivery and a second configuration for placement, in the second configuration the tube having an expanded configuration, the struts extending outwardly so that a distal region of the struts has a greater dimension and the struts defining a space therebetween, a mesh material supported by the struts and providing a blocking member to block blood clot migration from the appendage.

12. The device of claim 11, wherein the mesh material fills a substantial area of the space between the struts.

13. The device of claim 11, wherein the mesh material is in the form of a narrow strip attached to one or more of the struts.

14. The device of claim 11, wherein the mesh is attached to an outer surface of the struts.

15. The device of claim 14, wherein the mesh extends across a proximal region of the device.

16. The device of claim 11, wherein the retention member is composed of shape memory material.

17. A method for left atrial appendage occlusion comprising the steps of:
inserting into the left atrial appendage a sheath containing a retention member having a plurality of struts in a reduced profile position;
exposing the retention member from the sheath to enable it to expand to engage a wall of the left atrial appendage;
subsequently inserting mesh material in situ within a space between the plurality of struts; and
withdrawing the sheath to leave the retention member in the left atrial appendage so the mesh material fills the space within the retention member to block blood clot migration.

18. The method of claim 17, wherein the retention member has a plurality of shape memory struts and the step of exposing the retention member enables the struts to move toward a shape memorized position.

19. The method of claim 17, wherein the retention member has appendage engaging members to secure the retention member within the appendage.
